(19) United States
(12) Patent Application Publication
(10) Pub. No.: US 2025/0288765 A1
(43) Pub. Date: Sep. 18, 2025

(54) MEDICAL DEVICE FOR VENTILATING A LIVING BEING, PROCESS AND COMPUTER PROGRAM FOR OPERATING A MEDICAL DEVICE

(71) Applicant: Drägerwerk AG & Co. KGaA, Lübeck (DE)

(72) Inventor: Tobias GLAW, Lübeck (DE)

(21) Appl. No.: 19/081,083

(22) Filed: Mar. 17, 2025

(30) Foreign Application Priority Data
Mar. 18, 2024 (DE) 10 2024 107 611.9

(51) Int. Cl. A61M 16/00

(52) U.S. Cl. CPC A61M 16/024; A61M 2016/0018; A61M 2016/0027; A61M 2016/0036

(57) ABSTRACT

A process carries out an occlusion maneuver (100) and a medical device for ventilating a living beings incudes features with an operating function to carry out the occlusion maneuver (100) to determine an inspiratory pressure plateau (P_plat.) (111). A computer program or computer program product may carry out at least some of the process. During and after carrying out the occlusion maneuver, a signal analysis (110) can be used to determine whether a stable and reliable inspiratory pressure plateau (P_plat.) (111) could be determined as a result of the occlusion maneuver (100).

### CROSS REFERENCE TO RELATED APPLICATIONS

This application claims the benefit of priority under 35 U.S.C. § 119 of German Application 10 2024 107 611.9, filed Mar. 18, 2024, the entire contents of which are incorporated herein by reference.

### TECHNICAL FIELD

The present invention pertains to a medical device configured for ventilating a living being or a patient, such as an anesthesia device, in particular a ventilator in the form of an intensive care ventilator, a home ventilator, a mobile emergency ventilator or a ventilator for ventilating premature and/or newborn babies, infants and children, with an operating function for carrying out (executing) an occlusion maneuver to determine an inspiratory pressure plateau. Ventilators are often also referred to as respirators or ventilators.

In addition, the invention relates to a process for carrying out an occlusion maneuver with determination of an inspiratory pressure plateau, as well as to a computer program or computer program product for carrying out such a process.

### BACKGROUND

During ventilation, certain ventilation parameters such as ventilation pressures (P_insp, PEEP), ventilation frequency (RR), tidal volume (VT), I:E ratio, minute volume (MV) are set and continuously monitored by sensors. These ventilation parameters characterize both the patient's situation and the operating situation of the ventilator and its settings. In addition, it is often of interest to obtain information on how the patient's situation has changed during the course of treatment. Special maneuvers are often used for this purpose, some of which are performed manually by the user and some of which can also be supported by the ventilator. These maneuvers make it possible to determine certain characteristics of the patient from time to time.

Here are three examples of maneuvers:
RSVT maneuver (Respiratory Systolic Variation Test);
Occlusion maneuver;
P0.1-Maneuver.

An RSVT maneuver is used to determine the characteristics of a patient's cardiological system, in particular to obtain information about the patient's fluid balance. In everyday clinical practice, volume therapy for ventilated patients can be individually organized and/or controlled based on the results of the RSVT maneuver. During an RSVT maneuver, the ventilation device automatically increases the ventilation pressure in stages. These pressure levels of the ventilation pressure are increased simultaneously with arterial blood pressure measurement from a physiological patient monitor and the influence of the ventilation pressure on the blood pressure is determined and from this it is established whether the patient would benefit from the administration of additional fluid or not.

A P0.1 maneuver is used to determine the respiratory muscle function of a patient. A P0.1 maneuver is performed by occluding the airway for 0.12 seconds after a complete expiration at the start of the inspiratory phase while the patient breathing is at rest for a time duration of 0.12 seconds and determining the occlusion pressure 0.1 seconds after the start of inspiration. The occlusion pressure P0.1 is a reference value for the mean inspiratory pressure (P_insp.) that must be applied during each breath and thus characterizes both the load of the inspiratory muscles and the central respiratory drive. The P0.1 is also dependent, among other things, on the pressure transmission from the inspiratory muscles to the mouth and the extent of ventilation. Multiple repetitions, e.g. five repetitions, may be necessary to obtain an evaluable database.

In contrast to the P0.1 maneuver, the occlusion maneuver is not performed at the beginning of inspiration, but at or towards the end of the inspiration phase. The occlusion maneuver creates a situation in which ventilation is paused, as it were. Such a snapshot allows the user to evaluate the course (curve) of the airway pressure over the duration of the occlusion maneuver and use the curve to estimate an indicator of the work of breathing or the expansion of the patient's lungs.

### SUMMARY

It is an object of the invention to provide an operating function for a medical device—in particular for a ventilator—for ventilating a living being, which makes it possible to determine an indicator of a state of the lungs of the living being during inhalation.

A further object of the present invention is to provide a process with an operating function for operating a medical device for ventilating a living being in order to determine an indicator of the condition of the lungs of the living being.

A further object of the present invention is to provide a computer program or computer program product which enables an indicator of the condition of the lungs of the living being to be determined

These and other objects are addressed with features according to the invention for ventilating a living being and for carrying out an occlusion maneuver to determine an inspiratory pressure plateau, with a medical device comprising a line system; a breathing system with a conveying unit, a sensor system and a control unit operatively connected to the breathing system and the sensor system. The control unit with the connected features is configured to carry out ventilation and during a phase of an inspiration and/or following the phase of the inspiration to carrying out an occlusion maneuver with a closure of the breathing system for a predetermined time duration of an occlusion phase and to continue to carry out ventilation after the predetermined time duration of the occlusion phase or after termination of the occlusion phase. During the predetermined time duration of the occlusion phase, no quantities of inspiratory breathing gas mixture pass from the medical device to the living being and no quantities of expiratory breathing gas mixture pass away from the living being. The control unit is configured to determine the inspiratory pressure plateau based on the measured inspiratory pressure values acquired during the occlusion maneuver by means of a signal analysis carried out during the occlusion maneuver. The control unit carries out ventilation of the living being with the conveying unit, the breathing system and the line system after the occlusion phase. Ventilation may also continue immediately upon termination of the occlusion phase. The control unit generates an output signal, such as a display signal with an inspiratory pressure plateau indication of the determined inspiratory pressure plateau during the occlusion phase or with a termination indication that no reliable result of the inspiratory pressure plateau could be expected in the predetermined time duration of the occlusion phase. The output signal is generated essentially in real time, particularly during the occlusion phase or at the conclusion or termination of the occlusion phase. The control unit is configured to consider several conditions of terminating the already running occlusion maneuver in order not to burden the patient with an unpleasant type of breathing in cases no reliable result of the inspiratory pressure plateau could be expected in the predetermined time duration of the occlusion phase. In this way, the predetermined time duration of the occlusion phase may be made short, in view of the quick determination of the inspiratory pressure plateau during the occlusion phase or the quick termination of the occlusion phase when no reliable result of the inspiratory pressure plateau is to be expected. During the occlusion maneuver/occlusion phase the control unit may provide the display signal so as to indicate the time remaining until completion of the occlusion maneuver and/or the occlusion phase to keep the user informed about the ongoing time of the maneuver or the remaining time until normal end of the maneuver in case of normal operating the maneuver with the intended typical duration of time.

The object of providing an operating function for a ventilator is attained by a medical device—in particular a ventilator—with features according to the invention. The occlusion maneuver creates values and a pressure curve over time, which could not be achieved during normal ventilation. The control unit processor analyzes a current type of the pressure curve by data analysis to calculate the results with several scenarios as curve-criterions for determining the inspiratory pressure plateau on a high reliability level to indicate that the values, which have been achieved during the occlusion maneuver, are reliable for use to determine the inspiratory pressure plateau. The processor combines all the results of the scenarios in order to be prepared for determining the inspiratory pressure plateau. Only if all curve-criterions are met will the processor determine the inspiratory pressure plateau as an important and reliable value and important information for the clinician/medical doctor, which is indicating the physiological situation of the individual patient. Such a reliable value is provided as a signal output that may be displayed and then can be used for adapting the settings of the ventilator for further treatment of the patient. The device of the invention allows the controlled ventilation to be carried out before and after the controlled occlusion maneuver so as to provide real time information with minimal patient discomfort. The control unit may be configured to carry out the ventilation of the living being with the control of the conveying unit, the breathing system and the line system by controlling at least one of the predetermined parameters comprising duration of one phase of an inspiration, duration of one phase of an expiration, inspirational pressure, tidal volume, with changes to at least one of the predetermined parameters based on the determination of the inspiratory pressure plateau and/or display of determined inspiratory pressure plateau data for therapeutic treatment.

This object for a process for determining an indicator for a condition of the lungs of a living being is attained with features according to the invention and by a process with features according to the invention.

Furthermore, the process can also be provided as a computer program or a computer program product with features according to the invention, so that the scope of protection of the present application also extends to the computer program product and the computer program.

Features and details which are described in connection with the process according to the invention or the computer program product and the computer program naturally also apply in connection with and with regard to the device and vice versa, so that with regard to the disclosure of the individual aspects of the invention, mutual reference is or can be made.

Advantageous embodiments of the invention result from the subclaims and are explained in more detail in the following description with partial reference to the figures. Features and details that are described in connection with the medical device or ventilator according to the invention naturally also apply in connection with the process according to the invention and vice versa, so that with regard to the disclosure of the individual aspects of the invention, mutual reference is or can be made.

A medical device, in particular a ventilator for ventilating a living being or a patient, is configured to perform and configure ventilation.

The living being or patient is connected to the ventilator by means of a line system (tube system/breathing gas guide system), usually in the form of ventilation tubes (breathing tubes), and a patient connection element, the so-called Y-piece. The Y-piece is usually connected to an endotracheal tube for connection to the patient's tracheal tract. The Y-piece makes pneumatic contact with an inspiratory ventilation tube and provides the patient with quantities of gas that flow from the ventilator to the patient via the endotracheal tube for inhalation. The Y-piece pneumatically contacts an expiratory ventilation tube and delivers gas flowing from the endotracheal tube to the ventilator as the patient exhales. The inspiratory and expiratory ventilation tubes together with the Y-piece and the endotracheal tube thus form the line system for connection to the ventilator.

In conventional embodiments, the medical device has a control unit, a gas conveying unit and a breathing system (respiratory system) and is supplied with oxygen and medical air via a gas supply. The gas supply has suitably configured elements to provide the medical device or ventilator with sufficient quantities of a gas mixture. The gas mixture advantageously consists of an adjustable mixture of air and oxygen in order to enable ventilation of the patient with inhaled gases with concentrations above 21% oxygen. The gas supply is usually realized by means of a central gas supply (ZV), alternatively a gas conveying (delivery) unit, for example as a blower drive, compressor drive, or also a local compressed gas supply at the ventilator by means of cylinders can form the gas supply suitably and provide it to the ventilator.

In the operation of ventilators, the control unit is configured to perform and control with control and/or regulation of automated ventilation, i.e. for example control of pressure-controlled or volume-controlled ventilation. The control unit can also be configured to control and/or coordinate supportive forms of ventilation on the basis of measured values of ventilation pressures, inspiratory and/or expiratory flow rates or volumes (volume flow/volume rate of flow). Furthermore, the control unit is configured to control and/or manage alarm functions of the ventilator, including in particular threshold value monitoring of inspiratory and/or expiratory ventilation pressures, threshold value monitoring of gas concentrations, threshold value monitoring of expiratory flow rates.

An inspiratory branch of the breathing system supplies the gas volumes provided by the gas supply by means of a gas conveying unit, for example in the form of a radial blower, via a pneumatic interface to the inspiratory ventilation tube for conveyance (delivery) to the patient as an inhaled gas mixture.

An expiratory branch of the breathing system discharges into the environment (surroundings) in a controlled manner quantities of exhaled gas exhaled by the patient and carried away by the patient via the expiratory ventilation tube and flowing into the breathing system via a pneumatic interface.

The interfaces between the breathing system and the ventilation tubes are configured with—usually passive—non-return valves in such a way that the gas flows are directed in the breathing system and the line system are directed.

As explained above and below the so-called occlusion maneuver is used in everyday clinical practice to determine the current lung condition of the living being, to which the living being makes an independent contribution during ventilation, and the user subsequently performs a visual-manual evaluation of the course of the inspiratory pressure curve or airway pressure acquired (captured/recorded) during the occlusion maneuver. In particular, an inspiratory plateau pressure (P_Plat.) is observed during an occlusion phase in order to derive a characteristic pressure, the so-called “driving pressure”. Today, a measuring catheter in the esophagus is usually used to acquire the total driving pressure.

The addition of the positive pressure available at the ventilator during ventilation to the negative pressure measured with the measuring catheter results in the total “driving pressure” when using this catheter process. The pressure in the esophagus corresponds approximately to the pleural pressure when measured appropriately and therefore reflects the patient's respiratory effort quite accurately. In a preferred process for determining this value without using an esophageal measurement catheter, an inspiration (inhalation) is artificially prolonged until the patient neither inhales nor exhales and the respiratory muscles are therefore ideally passive. This results in an increase in pressure in the patient's lungs, as the total volume, i.e. the volume of gas moved by the ventilator and the volume moved by the patient himself/herself at a defined distensibility of the patient's lungs, is usually referred to as compliance, leads to a higher total pressure in the lungs when the respiratory muscles relax.

According to Formula 1, the driving pressure is calculated as a quotient of tidal volume and compliance:

$$Δ P = VT / C . \qquad Formula 1$$

With regard to the physical aspects of ventilating living beings, it should be noted that modern ventilators are able to determine properties of the line system or breathing line system such as resistance and compliance and possible leaks during a device self-test and then take them into account in further ventilation operation.

The basic idea of the invention is to determine or calculate an inspiratory plateau pressure (P_Plat.) from a pressure curve determined by means of the occlusion maneuver.

A control unit is arranged, provided and suitably configured in the medical device—in particular in the ventilator—for ventilating a living being in order to implement a metrological determination of this indicator or the “driving pressure” during the occlusion maneuver. This control unit is configured to perform automated ventilation with control and temporal coordination of inhalation (inspiration phases) and exhalation (expiration phases) of flow rates (Flow), volumes (Vt, MV), pressure levels such as inspiratory pressure (P_insp.) and positive end-expiratory pressure (PEEP) and to control the corresponding suitably configured actuators by means of signal and data lines.

The actuators in the ventilator that are controlled and actuated by the control unit are in particular the gas conveying unit, such as a radial blower or a piston pump, and controllable dosing valves, such as an expiratory valve.

In addition, the control unit is configured to acquire data from a sensor system with at least one pressure sensor and at least one flow rate sensor and to incorporate this data into the coordination and control of the automated ventilation. This control unit is also configured to control the occlusion maneuver and controls the actuators towards the end of the inhalation phase or beyond the end of the inhalation phase in such a way that the patient is not able to inhale or exhale for the duration of the occlusion maneuver. For the duration of the occlusion maneuver, the patient only has the option of exchanging gas with the volumes of the line system with inspiratory and expiratory ventilation tube. This means that the patient can breathe additional amounts of gas into the volumes of the line system from the lungs, which can lead to an increase in pressure in the line system, and can also breathe a certain amount of gas into the lungs from the volumes of the line system, which can lead to a reduction in pressure in the line system.

The control unit is suitably configured and provided to implement the basic idea described above in a medical device configured to ventilate a living being or a patient and is correspondingly extended by functionalities according to the invention to perform the occlusion maneuver

The control unit uses the occlusion maneuver to determine an inspiratory plateau pressure (P_Plat.) from the determined pressure curve. The control unit can derive the driving pressure as an indicator of the total work of breathing or ventilation work from the inspiratory plateau pressure (P_Plat.) determined.

A medical device according to the invention—in particular a ventilator—is configured to ventilate a living being or patient and comprises a control unit, a breathing system, a line system and a sensor system.

The inspiratory and expiratory ventilation tubes together with the Y-piece, the endotracheal tube, form a system of lines (line system) for connecting the ventilator to the living being or patient.

The breathing system is pneumatically connected to the line system by means of interfaces.

The breathing system has means for controlled conveyance and supply of an inspiratory quantity of a breathing gas mixture to the living being. An inspiratory branch of the breathing system provides controlled quantities of the breathing gas mixture, for example by means of a gas conveying unit in the form of a radial blower and by means of a pneumatic interface in the inspiratory ventilation tube for the patient.

The breathing system also has a means for a controlled guidance of an expiratory quantity of the breathing gas mixture. By means of a pneumatic interface, quantities of gas exhaled by the living being into the expiratory ventilation tube are discharged in a controlled manner via an expiratory branch of the breathing system or the line system, for example by means of an expiratory valve. The breathing system is thus configured to guide and control quantities of inhaled and exhaled gases within the medical device and to provide them to the living being or patient via the line system. For this purpose, the breathing system has means for conveying and guiding quantities of a breathing gas mixture with a controlled supply of an inspiratory quantity of the breathing gas mixture and means for a controlled guidance of an expiratory quantity of the breathing gas mixture. The conveying means can be configured as a gas conveyance (delivery) and metering unit, for example as a gas conveying unit in combination with a controllable proportional valve as an inspiratory valve. The means for guidance can be configured as an actively controllable expiration valve.

The interfaces to the ventilation tubes can be configured with—usually passive—non-return valves in order to predetermine the flow directions of the gas volumes in the breathing system and in the line system.

The medical device, according to the invention, provides an operating function with a coordination of an occlusion maneuver in order to perform a maneuver in the form of an occlusion maneuver, which enables an inspiratory pressure plateau (P_plat.) to be determined.

The line system is configured to supply breathing gas to the living being and to guide breathing gas away from the living being.

The breathing system is pneumatically connected to the line system by means of at least one interface or two interfaces. The line system is configured, for example, as a two-tube system with an inspiratory ventilation tube and an expiratory ventilation tube. Single-tube systems are often used, particularly in emergency ventilator configurations, in which the quantities of inhaled gases can be supplied to the patient via an inhalation tube and exhalation can take place via an exhalation valve controlled by the control unit, which is arranged close to the patient's mouth area. The breathing system can be configured as a breathing system of a ventilator or breathing system in a hospital intensive care unit (ICU), through which quantities of inhaled gases are provided to the patient by means of the line system via an inspiratory branch of the breathing system and quantities of exhaled gases can flow out directly at the patient or by means of recirculation through the line system to the ventilator and via an expiratory branch of the breathing system to the environment. The breathing system can also be configured as a line system of an anesthesia device or anesthesia system for carrying out inhalation anesthesia, through which quantities of inhalation gases are provided to the patient by means of the line system and quantities of exhalation gases can flow back through the line system to the anesthesia device or anesthesia system and circulate in the line system with components for carbon dioxide absorption and can be provided again as new inhalation gas.

The sensor system has at least one pressure sensor, which is arranged on the breathing system or on the duct system in such a way that it continuously detects an inspiratory pressure level or a time course of the inspiratory pressure level and provides it to the control unit as inspiratory pressure measurement values. The at least one pressure sensor can, for example, be configured as an inspiratory pressure sensor, which can be arranged on an inspiratory branch of the breathing system. The at least one pressure sensor can, for example, be configured as an expiratory pressure sensor, which can be arranged on an expiratory branch of the breathing system. The at least one pressure sensor can, for example, be configured as a pressure sensor close to the patient or a patient pressure sensor, which can be arranged on the patient connecting element (Y-piece) as well as on the inspiratory and expiratory ventilation tube.

The sensor system can, particularly in a preferred embodiment, have at least one flow rate sensor, which is arranged on the breathing system or on the line system in such a way that at least one measured value of a flow rate is continuously acquired and made available to the control unit.

The at least one flow rate sensor can, for example, be configured as an inspiratory flow rate sensor, which can be arranged on an inspiratory branch of the breathing system. The at least one flow rate sensor can, for example, be configured as an expiratory flow rate sensor, which can be arranged on an expiratory branch of the breathing system. The at least one flow rate sensor can, for example, be configured as a flow rate sensor close to the patient or a patient flow rate sensor, which can be arranged on the patient connecting element (Y-piece), as well as on the inspiratory and expiratory ventilation tube.

The control unit is configured to perform ventilation of the living being with the means for controlled supply and guidance of quantities of breathing gas mixture to the breathing system and the line system and to control at least one of the predefined parameters, such as a duration of a phase of an inspiration (Ti), a duration of a phase of an expiration (Te), an inspiratory pressure (P_insp.), a tidal volume (Vt), a ventilation frequency (RR). The control unit can be configured to use both the expiratory pressure sensor and/or the inspiratory pressure sensor and/or the patient pressure sensor for carrying out ventilation and/or coordinating the occlusion maneuver to determine the inspiratory pressure (P_insp.) depending on the situation. The control unit can be configured to use both the expiratory flow rate sensor and/or the inspiratory flow rate sensor and/or the patient flow rate sensor for measuring the flow rate in order to perform ventilation and/or coordinate the occlusion maneuver.

The control unit is also configured to perform, control and coordinate ventilation of the living being with the sensor system, the breathing system, the line system, the means for controlled dosing of the inspiratory breathing gas quantity and the expiratory valve. The control unit is configured to receive data or signals from the sensors, to process them by means of signal processing (A/D conversion) and/or signal amplification (OP amps) and/or signal filtering, to process the signals by means of a computing unit and to carry out evaluations with the processed data.

The computing unit can, for example, be configured as a processor unit (μP, μC) with an associated data memory (RAM, ROM). For example, the processing unit—and therefore also the control unit—can be configured using a program code and programmed to determine the current operating status of the medical device or situations of ventilation, inhalation, exhalation in interaction and/or interaction with the patient from data or signals from the sensors.

According to the invention, the control unit is configured to perform an occlusion maneuver with a closure of the breathing system for a predetermined time duration of an occlusion phase with the means for controlled dosing of the inspiratory and expiratory quantities of breathing gas mixture on the living being during the phase of an inspiration (Ti) and/or following the phase of the inspiration (Ti). For the predetermined time duration of the occlusion phase, no quantities of inspiratory breathing gas mixture can pass from the medical device to the living being and no quantities of expiratory breathing gas mixture can pass away from the living being—for example into the environment

The occlusion maneuver can and should now be explained in the context of an “inspiration hold” maneuver, or “inspiratory breath hold” or “inspiratory hold maneuver”, wherein it is clear from the term itself that an inspiratory phase can be held, i.e. extended beyond a typical time duration. During such an inspiration hold maneuver, the expiration valve is activated by the control unit after the already completed inspiration phase in such a way that the expiration valve remains closed for a time duration of 1 second to 40 seconds. The inspiration hold maneuver can be used to determine the inspiratory pressure in the lungs of the living being,

The term occlusion maneuver as well as the definition of the occlusion phase are broader in the sense of the present invention than previously explained for the inspiration-hold maneuver.

The occlusion phase does not necessarily represent a time duration exclusively towards the end of the inspiratory phase or after the end of the inspiratory phase, but can begin with the occlusion of the breathing system after half or two thirds of the ventilation settings activated on the ventilator for the inspiratory phase T_insp. The ventilation settings such as respiratory rate (RR) and expiratory-to-inspiratory ratio (I:E ratio) also affect the duration of the inspiratory phase T_insp, so that the duration of the inspiratory phase can also result indirectly from other settings.

The occlusion phase can therefore also be completed during the time duration of the inspiration phase or not extend beyond the end of the inspiration phase. The predetermined time duration of the closure of the breathing system, i.e. the time duration of the occlusion and thus the duration of the occlusion phase, can be selected in a range from 1 second to 5 seconds, preferably 2.0 seconds. The control unit may monitor this selected time duration of the occlusion maneuver and generate the display signal so as to indicate the time remaining until completion of the occlusion maneuver and/or the occlusion phase to keep the user informed about the ongoing time of the maneuver or the remaining time until normal end of the maneuver in case of normal operating the maneuver with the intended typical duration of time. The generated display signal may also indicate a termination of the occlusion maneuver, keeping the user informed in case of terminating of the already running occlusion maneuver in order to not burden the patient with a unpleasant type of breathing in cases no reliable result of the inspiratory pressure plateau could be expected in the selected time duration of the occlusion maneuver. With the medical device (ventilator) according to the invention, the control device generates display signals including providing and/or displaying measured values or measurement curves of the sensor system, events and alarm situations, during ventilation, and also during the occlusion maneuver and/or the occlusion phase. The generated and or displayed signals may include one or more of the measured values or measurement curves, the set duration of the occlusion phase, the remaining time until normal end of the maneuver, the determined maneuver inspiratory pressure plateau, the termination information if there is termination and/or settings information for continued ventilation.

According to the invention, the control unit is further configured to determine an inspiratory pressure plateau (P_plat.) on the basis of the measured inspiratory pressure values acquired during the occlusion maneuver by means of a signal analysis. The signal analysis is preferably performed over a defined time period—also referred to as a time window—after the start of the occlusion maneuver, for example over a time window of 0.2 seconds to 0.8 seconds, preferably 0.4 seconds. For a practical realization of the signal processing in a medical device by means of the control unit, it can be advantageous that the measured pressure values are smoothed and/or filtered (cleaned) of interfering components by means of suitable data preprocessing such as averaging, median formation and/or suitable signal filtering using low-pass filters. On the one hand, the control unit itself may have suitable means for this, but the medical device may also have components for signal pre-processing which can perform the tasks mentioned and supplement the control unit.

In a further preferred embodiment, the control unit can be configured to include the elasticity of components of the line system in the determination of the inspiratory pressure plateau (P_plat.). The line system is usually formed from elastic ventilation tubes, which are elastic and can therefore partially buffer or compensate for pressure increases in the breathing system, line system and lungs of the living being during ventilation and/or during the occlusion maneuver by elastic deformation. Therefore, changes in pressure during the occlusion maneuver are only partially caused by inhalation efforts and/or the relaxation of the respiratory muscles; rather, the properties of the line system also influence the extent of such pressure changes as part of the physical conditions.

In current ventilators, it is common practice to perform compliance compensation during ventilation, as the volume delivered into the breathing circuit (tubes) is not always identical to the actual volume that the patient receives. The difference between the two volumes is essentially caused by the compliance of the breathing circuit and line system. This is usually determined by checking the properties of the tubing system or line system, in particular the elasticity or compliance of the tubing system during a device self-test with compliance test and resistance test immediately after commissioning or as part of a leakage test in the anesthesia or ventilation device. During a leakage test, it is determined whether and to what extent there is a leak in the line system or the connection to the patient. During a compliance test, it is determined whether and to what extent the line system is capable of a change in volume when the ventilation pressure changes. The extent of such a change is a characteristic of the line system or the breathing line system and is referred to as tube compliance. During a resistance test, the magnitude of the pressure drop across the line system resulting from changes in the flow rate (flow rate=dV/dt) is determined. The extent of such a pressure drop is a property of the line system or the breathing circuit and is referred to as tube resistance. Tube resistance and tube compliance depend on the diameter, length and material of the line system or tube system. By means of the device self-test with leakage test, compliance test and resistance test, the respective configuration of ventilator, line system and a patient interface configured as a patient connecting element (Y-piece) intended for use on the patient can be calibrated with regard to leakage, resistance and compliance. During subsequent ventilation operation, the control unit can balance the tube resistance and tube compliance when dosing gas volumes so that the patient actually receives the intended gas volumes during ventilation. In this preferred embodiment, the control unit can use previously determined and thus known compliance of the tubing system as elasticity of the line system and include it in the determination of the inspiratory pressure plateau (P_plat.) according to formula 1.

In a preferred embodiment, the sensor system can have a further pressure sensor, which can be arranged on the breathing system or on the line system in order to continuously detect an expiratory pressure level or a time course of the expiratory pressure level and provide it to the control unit as expiratory pressure measurement values. Like the at least one pressure sensor, the additional pressure sensor can, for example, be configured as an expiratory pressure sensor, which can be arranged on an expiratory branch of the breathing system.

The additional pressure sensor can also be configured as an inspiratory pressure sensor, for example, which can be arranged on an inspiratory branch of the breathing system. The additional pressure sensor can, for example, be configured as a pressure sensor close to the patient or a patient pressure sensor, which can be arranged on the patient connecting element (Y-piece) on the inspiratory and expiratory ventilation tube. The control unit can be configured to use both the expiratory pressure sensor and/or the inspiratory pressure sensor and/or the patient pressure sensor to determine an expiratory pressure (P_exsp.)—in particular an end-expiratory pressure (PEEP)—depending on the situation for carrying out ventilation and/or coordinating the occlusion maneuver.

In this preferred embodiment, the control unit can also be configured to calculate the inspiratory pressure plateau (P_plat.) and the end-expiratory pressure (PEEP) on the basis of the inspiratory pressure.
an indicator (P_driv.) for a total work of breathing (respiratory work) and/or
an indicator (P_spon.) for the proportion of spontaneous respiration of the living being in the total work of breathing and/or
an indicator (P_vent.) for the proportion of the medical device or ventilator in the total work of breathing and/or
an indicator (C_stat.) for the static distensibility of the lungs of the living being.

The indicator (P_driv.) for a total work of breathing can be determined from the relationship between the inspiratory pressure plateau (P_plat.) and the positive end-expiratory pressure (PEEP) according to the following formula 2:

$$P\_driv . = P\_plat . - PEEP \qquad Formula 2$$

The indicator (P_spon.) for the proportion of spontaneous breathing of the living being in the total work of breathing can be determined from the relationship between the pressure level (P_plat.) and the inspiratory pressure (P_insp.) according to the following formula 3:

$$P\_spon . = P\_plat . - P\_insp . \qquad Formula 3$$

The indicator (P_vent.) of the share of the medical device or ventilator in the total work of breathing can be determined from the relationship between the inspiratory pressure (P_insp.) and the positive end-expiratory pressure (PEEP) according to the following formula 4:

$$P\_vent . = P\_insp . - PEEP \qquad Formula 4$$

In Formula 3 and Formula 4, the inspiratory pressure (P_insp.) is a pressure value measured at a point in time before the occlusion maneuver and effectively provided at the patient.

The determination of the (C_stat.) for a static distensibility (static compliance) of the lungs of the living being can be carried out with knowledge of the volumes given during the inspiratory phase according to the following formula 5:

$$C\_stat . = V\_insp . / P\_driv . \qquad Formula 5$$

The inspiratory volume (V_insp.) is the volume that is supplied to the patient during the inspiratory phase and is also measured. To acquire the volume (V_insp.), the at least one flow rate sensor can be used by the control unit, which can be arranged on an inspiratory or expiratory branch of the breathing system or as a flow rate sensor close to the patient or patient flow rate sensor on the patient connecting element (Y-piece) of the line system.

In a further preferred embodiment, the control unit can be configured, to determine the inspiratory pressure plateau (P_plat.) by applying a statistical process, in particular by applying a plurality of sectional linear, non-linear or polynomial regressions over the signal course (signal curve) of the inspiratory pressure measurement values during the occlusion phase. The regression is preferably carried out after a certain waiting time of 0.25 seconds to 0.75 seconds over the duration of the occlusion phase.

In a further preferred embodiment, the control unit can be configured, to determine the inspiratory pressure plateau (P_plat.) by using an analytical process, in particular by using a continuous determination of a gradient over the signal course of the inspiratory pressure measurement values during the occlusion phase. For this purpose, the control unit can differentiate the signal course by means of 1-st derivative of the pressure P′(t)=dP/dt; 2-nd derivative of the pressure P″(t)=d²P/dt²; 3rd derivative of the pressure P′″(t)=d³P/dt³) of the signal course of the inspiratory pressure measurement values starting with the closure of the breathing system in order to determine characteristics in the signal course, in particular slopes, signs of the slopes, zero points, maxima, minima, inflection points or saddle points in the signal course and thus to identify a phase of an essentially constant inspiratory pressure plateau in contrast to phases with a pressure increase or pressure drop and/or phases with signal fluctuations. A constant plateau can be identified, for example, by evaluating the third derivative if this is found to fluctuate around zero ([f′=0] & [f″=0] & [f′″≠0]->saddle point) for a predominant number of values for a longer time interval. Phases with signal fluctuations, on the other hand, show an alternation of maxima and minima ([f′=0] & [f″≠0]) in the signal course at short intervals.

In a further preferred embodiment, the control unit can be configured to incorporate mathematical processes—in particular processes of pattern comparison or processes of trend analysis—of the inspiratory pressure measurement values with comparative data to determine the inspiratory pressure plateau (P_plat.). For this purpose, the control unit can be assigned a data memory in which typical signal courses of inspiratory pressure measurement values during an occlusion maneuver are stored. Pattern matching processes can be used by the control unit to determine an indicator of similarity for different phases in the signal course of the current inspiratory pressure measurement values with the stored patterns. Before using such pattern comparisons, it can be advantageous to normalize the inspiratory pressure measurement values so that a comparison can be made with normalized typical signal courses of the data memory.

The mathematical processes of this preferred embodiment also include, in a broader sense, processes in which different forms of neural networks (single-layer and multi-layer feedforward networks, networks with feedback (recurrent networks)) or processes of so-called deep learning are used, which can be taught or trained in different ways (supervised or unsupervised learning, stochastic learning) to determine the inspiratory pressure plateau (P_plat).

In a further preferred embodiment, the control unit can be configured to evaluate (check) at least one criterion when determining the inspiratory pressure plateau (P_plat.) during the occlusion phase. The at least one criterion can include
a maximum time duration (t_rise_max.) of a rise time of the inspiratory pressure measurement values and/or
a minimum time duration (t_plat_min.) of a plateau of the inspiratory pressure measurement values and/or
a stability (P_stabil.) of the plateau of the inspiratory pressure measurement values can be included. To check the at least one criterion, the control unit evaluates the signal course of the inspiratory pressure measurement values during the occlusion phase by means of a signal analysis

The at least one criterion of a maximum time duration (t_rise_max.) of a rise time of the inspiratory pressure measurement values evaluates how long it takes until a rise to a stable inspiratory pressure plateau (P_plat.) occurs after the breathing system is closed. The control unit can use a regression analysis of the pressure signal or a differentiation (1-st derivative of the pressure P′(t)=ΔP/Δt), starting with the closure of the breathing system, to analyze the pressure signal to determine whether a predefined maximum duration of the rise time of the inspiratory pressure measurement values has not been exceeded. This can be implemented in practice, for example, by the control unit using a threshold value comparison to assess when the differentiated pressure signal P′(t) approaches zero, which indicates the end of the pressure rise and a transition to a pressure plateau.

Alternatively, the control unit can, for example, use a continuously repeating linear regression analysis according to the approach P(t)˜y(t)=a*t+b over a predetermined number of inspiratory pressure measurement values or a predetermined time duration with inspiratory pressure measurement values to evaluate when the factor a in the term y(t)=a*t+b tends towards zero, which indicates an end of the pressure increase with a flattening of the slope a and a transition to a pressure plateau. For implementation in practice, a predetermined maximum rise time (t_rise_max.) of less than 0.75 seconds to 1.25 seconds has proven to be advantageous.

The at least one criterion of a minimum time duration (t_plat_min.) of a plateau of the inspiratory pressure measurement values evaluates whether the pressure increase has been completed after the patient's breathing system or airways have been closed and a constant inspiratory pressure plateau (P_plat.) has been established. The control unit can use a regression analysis of the pressure signal or a differentiation (1st derivative of the pressure P′(t)=ΔP/Δt), starting with the closure of the patient's breathing system or airway, to analyze the pressure signal to determine whether a pressure plateau is present. This can be implemented in practice, for example, by the control unit determining that the differentiated pressure signal P′(t) is almost constant or constant at zero for a predetermined time duration, which indicates a constant pressure plateau. Alternatively, the control unit can, for example, use a continuously repeating linear regression analysis according to the approach P(t)˜y(t)=a*t+b over a predetermined number of inspiratory pressure measurement values or a predetermined time duration with inspiratory pressure measurement values to evaluate when and over what time duration the factor a in the term y(t)=a*t+b tends towards zero, which indicates a slope of almost zero (a=0). If, in addition, this gradient remains virtually unchanged for a predetermined time duration, this indicates a constant pressure plateau. For implementation in practice, a time interval in the range of 1.0 to 2.0 seconds has proven to be advantageous as the minimum predetermined time duration (t_plat_min.).

The at least one criterion of a stability (P_stable.) of the plateau of the inspiratory pressure measurement values can be implemented by the control unit in that, for example, a standard deviation or variance of the measurement values of the inspiratory pressure (P_insp.) or the measurement values of the airway pressure (P_aw) is determined during the duration of the pressure plateau and compared with a predetermined threshold value of a maximum permissible fluctuation of the inspiratory pressure measurement values (P_insp.) during the duration of the pressure plateau. For implementation in practice, a predetermined threshold value (P_stabil.) for a pressure fluctuation of the measured values of the inspiratory pressure (P_insp.) in a range of 0.4 mbar per second to 0.8 mbar per second has proven to be advantageous.

Such criteria can be applied, for example, by using a sequence of sectional linear regressions or linear regressions over predetermined regression periods for measured values of the inspiratory pressure (P_insp.) and can characterize the course of the measured values of the inspiratory pressure (P_insp.) as parameters during the time duration of the occlusion phase. The measured values of the inspiratory pressure (P_insp.) used as the regression period for the linear regression preferably correspond to a time interval of 0.2 seconds to 0.6 seconds, preferably 0.4 seconds. Carrying out the linear regression over the duration of the occlusion phase results in a large number of straight line segments, which can be compared, analyzed and interpreted individually or with each other in order to evaluate and interpret the course and shape of the inspiratory pressure (P_insp.) and to determine whether and at what point in time a stable inspiratory pressure plateau (P_plat.) has been established after activation of the occlusion with closure of the breathing system and what pressure level the determined inspiratory pressure plateau (P_plat.) exhibits.

The control unit can be configured to determine suitable parameters as various options for checking the above criteria. The following examples are given here as a selection:
i. a parameter K1 (slope index), which indicates the slope of the respective line segments determined using linear regression;
ii. a parameter K2 (regression value index), which in each case indicates a difference in the measured values of the inspiratory pressure (P_insp.) used for linear regression in relation to the value on the straight line section approximated by means of linear regression;
iii. a parameter K3 (mean value index), which indicates a mean value of the values approximated on the basis of the linear regression.

The parameters K1, K2, K3 can—if advantageous also be normalized, inverse normalized and/or weighted for further analysis and simplification.

Furthermore, the parameters K1, K2, K3 can also be combined in a weighted or unweighted manner, from which an overall parameter KG can then be formed. This total parameter KG directly and/or indirectly indicates the inspiratory plateau pressure (P_plat.).

In a further preferred embodiment, the control unit can be configured to include a reliability indicator when determining the inspiratory pressure plateau (P_plat.) during the occlusion phase. Such a reliability indicator can be determined continuously during the occlusion maneuver. The reliability indicator can be determined—preferably with a time delay of, for example, 0.5 seconds to 2 seconds—starting with the start of the occlusion maneuver based on the measurement of the inspiratory pressure (P_insp.). To form the reliability indicator, a standard deviation of the measured values of the inspiratory pressure (P_insp.) can be determined over predetermined time duration of, for example, 1 to 2 seconds and repeated continuously after each regression period—for example, every 50 ms. For evaluation purposes, the reliability indicator can be compared with threshold values in order to evaluate the result of the determination of the inspiratory pressure plateau (P_plat.).

If the reliability indicator determined is below a predetermined threshold value of 0.6 mbar, for example, the measurement is considered successful, and the signal analysis is continued until the predetermined time duration of the occlusion phase has elapsed.

If the reliability indicator is below a further predetermined threshold value, the plateau pressure determination can be terminated directly and immediately according to a particularly preferred embodiment, since the continuous determination of the inspiratory pressure plateau (P_plat.) over the course of the occlusion phase has already produced a very good result. If the determined standard deviation of the measured values of the inspiratory pressure (P_insp.) or the measured values of the airway pressure (P_aw) falls significantly below the further predetermined threshold value of, for example, 0.3 mbar before the end of the second half of the occlusion maneuver, the occlusion maneuver can therefore be terminated prematurely, as the determined inspiratory pressure plateau (P_plat.) is already very reliable. The determination of the inspiratory pressure plateau (P_plat.) was therefore successful.

Another preferred embodiment can be formed by evaluating an exceedance of a threshold value by means of a comparison in order to terminate the occlusion maneuver prematurely. If, for example, during the second half of the occlusion maneuver the determined standard deviation of the measured values of the inspiratory pressure (P_insp.) or the measured values of the airway pressure (P_aw) permanently exceeds the predetermined threshold value and/or a special further threshold value of, for example, 0.9 mbar, the occlusion maneuver can also be terminated prematurely, as it cannot be expected that a reliable value of the inspiratory pressure plateau (P_plat.) can still be determined during the current occlusion maneuver. The determination of the inspiratory pressure plateau (P_plat.) was therefore not successful.

Premature termination of the occlusion maneuver offers the patient a considerable comfort advantage, as the airway closure lasts only as long as is actually required to establish a stable inspiratory pressure plateau (P_plat.). The medical device of the invention carries out ventilation before and after an occlusion maneuver. During the occlusion maneuver there is a continuous monitoring of the form of the pressure (increase of the slope, max. time until reaching the plateau, stability of the plateau). As noted, the control unit is configured for determining the inspiratory pressure plateau via the signal course of the inspiratory pressure measured values during the occlusion phase to apply or carry out a statistical methodology, in particular using a plurality of sectional linear, non-linear or polynomial regressions, or an analytical process, in particular using a continuous determination of a gradient over the signal course of the inspiratory pressure measured values, or mathematical processes of pattern comparison or trend analysis of the inspiratory pressure measured values with comparative data. The control unit is configured to evaluate, when determining the inspiratory pressure plateau during the occlusion phase, at least one criterion during the occlusion phase, wherein in the at least one criterion comprises a maximum time duration of a rise time of the inspiratory pressure measurement values and/or a minimum time duration of a plateau of the inspiratory pressure measurement values and/or a stability of the plateau of the inspiratory pressure measurement values. The control unit is configured to include a reliability indicator in the determination of the inspiratory pressure plateau during the occlusion phase. The control unit is configured to terminate the occlusion maneuver based on the verification of the reliability indicator. If a stable inspiratory pressure plateau cannot be expected by the result of analyzing the increase of the pressure slope and/or the standard deviation is higher than a threshold, in such scenarios the occlusion maneuver is stopped (terminated) and the normal ventilation will be continued. The user may start a next try and start the same maneuver again 1-2 hours later. As such there are several conditions of terminating the already running occlusion maneuver in order not to burden the patient with an unpleasant type of breathing in cases no reliable result of the inspiratory pressure plateau could be expected. In this way, the predetermined time duration of the occlusion phase may be made short, in view of the quick determination of the inspiratory pressure plateau during the occlusion phase or the quick termination of the occlusion phase when no reliable result of the inspiratory pressure plateau is to be expected.

In a further preferred embodiment, an input element can be arranged on the medical device, which is configured and intended to provide a signal to the control unit in order to cause the control unit to initiate an activation or start of the occlusion maneuver. Actuation of the input element by a user can cause activation of the occlusion maneuver and closure of the breathing system for a predetermined or maximum time interval. In an alternative embodiment, the actuation of the input element by the user can activate the occlusion maneuver and close the breathing system for the duration of the actuation of the input element. This allows the user to select and activate an automated sequence of the occlusion maneuver with a predetermined time duration as well as individually extend the time duration of the occlusion phase beyond the predetermined duration—for example, based on their own observations of curves of the flow and/or pressure curve on the ventilator.

According to a further aspect of the invention, a solution to the problem is provided by a process for determining an inspiratory pressure plateau.

In the process according to the invention, an occlusion maneuver is used to implement and enable the determination of the inspiratory pressure plateau in practice.

A process for determining an inspiratory pressure plateau (P_plat.) can be configured using a sequence of steps comprising the following steps:
Providing a data set which indicates a signal course of inspiratory pressure measurement values, wherein the inspiratory pressure measurement values correspond to a time duration of an occlusion phase,
Carrying out a signal analysis of the inspiratory pressure measurement values to determine the inspiratory pressure plateau (P_plat.).

The data set, which indicates a signal course of inspiratory pressure measurement values, can be provided in a data network by a medical device configured to ventilate a living being, and the signal analysis of the inspiratory pressure measurement values to determine the inspiratory pressure plateau (P_plat.) can take place in the data network, for example by means of a computing unit (server) suitably configured by programming.

A process according to the invention with an operating function for operating a medical device configured for ventilating a living being with determination of an inspiratory pressure plateau (P_plat.) can be configured by a sequence of steps comprising the following steps:
activation of means for controlled dosing of the inspiratory and expiratory quantities of breathing gas mixture in order to coordinate ventilation with a cyclical sequence of phases with inspiration and expiration,
activation of components of the medical device to cause occlusion of a breathing system during an inspiratory phase or at the end of an inspiratory phase for a predetermined time duration of an occlusion phase;
provision or acquisition of a signal course of pressure measurement values that indicate an inspiratory pressure level;
carrying out a signal analysis of the inspiratory pressure measurement values to determine the inspiratory pressure plateau (P_plat.).

This sequence of steps can, for example, be coordinated by a control unit of a medical device. The data set, which indicates a signal course of inspiratory pressure measurement values, can be provided in a data network by a medical device configured to ventilate a living being, and the signal analysis of the inspiratory pressure measurement values to determine the inspiratory pressure plateau (P_plat.) can take place in the data network, for example by means of a control unit or computing unit suitably configured by programming.

The aspects which have been described and explained in relation to definitions, modes of operation and scope with regard to the operating function, inspiration phase, occlusion phase and occlusion maneuver in the context of the description of the medical device according to the invention naturally also apply to the processes according to the invention.

In a preferred embodiment of the process, a data set is provided in a further step, which indicates a signal course of expiratory pressure measurement values and/or an end-expiratory pressure (PEEP), or a signal course of expiratory pressure measurement values is acquired. In a subsequent step, based on the inspiratory pressure plateau (P_plat.) and the end-expiratory pressure (PEEP), a signal analysis can be performed in this preferred embodiment to determine
an indicator (P_driv.) for a total work of breathing and/or
an indicator (P_spon.) for a share of spontaneous respiration of the living being of the total work of breathing and/or
an indicator (P_vent.) for a share of the medical device or ventilator in the total work of breathing and/or
an indicator (C_stat.) for a static distensibility of the lung of the living being.

The data set, which indicates a signal course of expiratory pressure measurement values, can be provided by the medical device by means of a metrological acquisition. The medical device can also provide the data set, which indicates a signal course of measured expiratory pressure values, in the data network.

The signal analysis based on the inspiratory pressure plateau (P_plat.) and the end-expiratory pressure (PEEP) can be carried out by a control unit of the medical device and/or in the data network by a computing unit that has been suitably configured by programming, for example. The determination
of the indicator (P_driv.) for a total work of breathing,
of the indicator (P_spon.) for a share of spontaneous respiration of the living being of the total work of breathing,
of the indicator (P_vent.) for a share of the medical device or ventilator of the total work of breathing,
of the indicator (C_stat.) for a static distensibility of the lung of the living being can, for example and/or preferably, be carried out as described—stating the corresponding relationships and/or formulae—for the corresponding embodiment of the medical device according to the invention, in particular the ventilator according to the invention.

In a preferred embodiment of the process, when carrying out the signal analysis to determine the inspiratory pressure plateau (P_plat.), an application of
statistical processes in embodiments of a variety of sectional regressions, such as linear, nonlinear or polynomial regressions or
analytical processes with continuous determination of a gradient over the signal course of the inspiratory pressure measurement values or
mathematical processes of pattern comparison of inspiratory pressure readings with comparative data
   can be performed. The signal analysis for determining the inspiratory pressure plateau (P_plat.) can, for example and/or preferably, be carried as described for the corresponding embodiment of the medical device according to the invention.

In a preferred embodiment of the process, at least one criterion can be evaluated (checked) when determining the inspiratory pressure plateau (P_plat.). The at least one criterion can include
a maximum time duration (t_rise_max.) of a rise time of the inspiratory pressure measurement values and/or
a minimum time duration (t_plat_min.) of a plateau of the inspiratory pressure measurement values and/or
a stability (P_stabil.) of the plateau of the inspiratory pressure measurement values.

The configuration and the inclusion of the criteria (t_rise_max.), (t_plat_min.), (P_stable.) in the determination of the inspiratory pressure plateau (P_plat.) can, for example and/or preferably, be carried out as described for the corresponding embodiment of the medical device according to the invention.

In a preferred embodiment of the process, a reliability indicator can be checked when determining the inspiratory pressure plateau (P_plat.).

The inclusion of the reliability indicator in the determination of the inspiratory pressure plateau (P_plat.) can preferably be carried out in a similar manner as described for the corresponding embodiment of the medical device according to the invention.

In a preferred embodiment of the process, properties of the line system, in particular an elasticity of components of the line system, can be included in the determination of the inspiratory pressure plateau (P_plat.). The inclusion of the properties of the line system in the determination of the inspiratory pressure plateau (P_plat.) can, for example and/or preferably, be carried out as described for the corresponding embodiment of the medical device according to the invention.

A further embodiment according to the invention is formed by a computer program or computer program product with a program code for determining an inspiratory pressure plateau. The computer program is non-transitory and fixed in tangible media. The computer program or the computer program product is suitably configured to carry out the process and is thus configured to determine the inspiratory pressure plateau (P_plat.). The program code can preferably be executed on a computer, a processor or a programmable hardware component. Steps, operations or processes of various processes described above may be executed by programmed computers or processors. Examples may also cover program storage devices (the tangible media), such as digital data storage media, which are machine-, processor- or computer-readable and encode machine-executable, processor-executable or computer-executable programs of instructions. The instructions perform or cause the performance of some, or all of the steps of the processes described above. The program storage devices may include or be, for example, digital storage devices, magnetic storage media such as magnetic disks and magnetic tapes, hard disk drives, or optically readable digital data storage media or other non-transitory tangible media. Further examples may also cover computers, processors or control units programmed to perform the steps of the processes described above, or (field) programmable logic arrays ((F)PLAs) or (field) programmable gate arrays ((F)PGAs) programmed to perform the steps of the processes described above. It is to be understood that the disclosure of multiple steps, processes, operations or functions disclosed in the description or claims is not to be construed as being in the particular order unless explicitly or implicitly stated otherwise, e.g. for technical reasons.

Further, in some examples, a single step, function, process or operation may include and/or be broken into multiple sub-steps, sub-functions, sub-processes or sub-operations. Such sub-steps may be included and form part of the disclosure of that single step, unless they are explicitly excluded.

In summary, the present invention makes it possible to determine an inspiratory pressure plateau (P_plat.) in a reliable manner, to determine the “driving pressure” (P_driv.) based on this as an indicator of a total work of breathing and to make it available to the user.

In the following, exemplary embodiments of the invention are explained in more detail with reference to the figures, without limiting the generality of the inventive concept. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic representation of a ventilator;

FIG. 2 is a schematic flow sequence of an occlusion maneuver;

FIG. 3 is a schematic flow sequence showing detailed aspects of the schematic sequence shown in FIG. 2; and

FIG. 4 is a view showing aspects of an evaluation of pressure signal courses.

### DESCRIPTION OF PREFERRED EMBODIMENTS

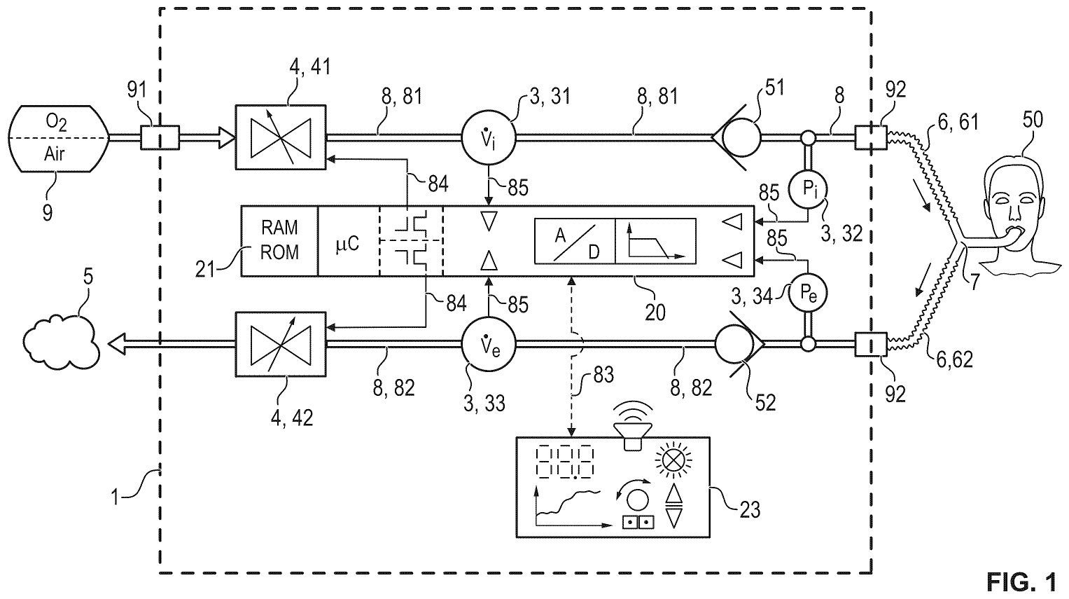

Referring to the drawings, FIG. 1 shows a schematic representation of a ventilator 1 with a gas supply 9 and a breathing system 8. A sensor system 3 with an inspiratory flow rate sensor 31, an inspiratory pressure sensor 32, an expiratory flow rate sensor 33 and an expiratory pressure sensor 34 is shown on (operatively connected to) the breathing system 8.

Actuators 4 with an inspiratory metering element 41—for example in the form of a gas conveying unit (blower) in combination with a proportional valve—and an expiratory valve 42 as well as passive non-return valves 51, 52 are shown in the breathing system 8, each in an inspiratory branch 81 and an expiratory branch 82.

The line system 6 connects the breathing system 8 via pneumatic interfaces 92 with an inspiratory ventilation tube 61 and an expiratory ventilation tube 62 by means of a patient connecting element (Y-piece) 7 to a patient 50 for a supply of quantities of breathing gases to the patient 50 and for a guidance of exhaled gases from the patient 50 to an environment 5.

The gas supply 9 provides the breathing system 8 in the ventilator 1 with medical air (Air) and oxygen (O₂) at a further pneumatic interface 91

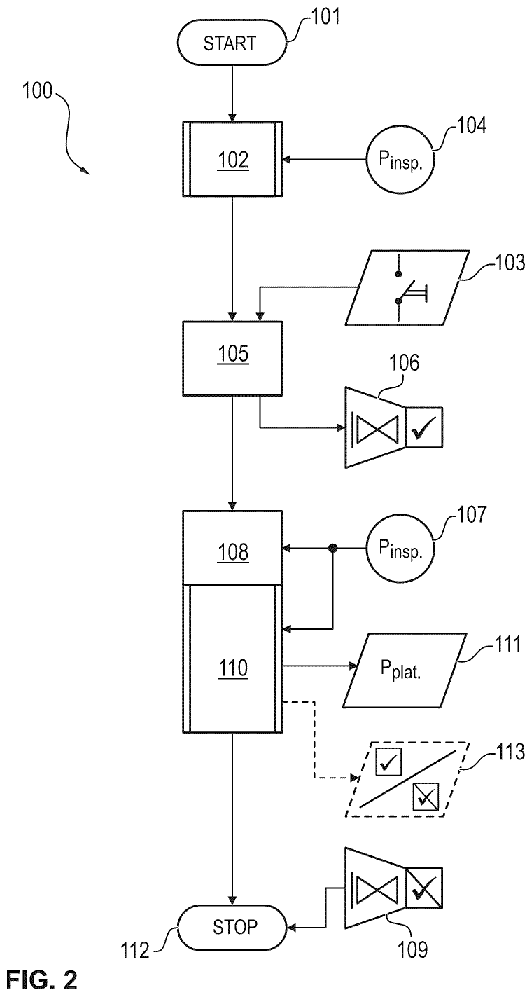

A control unit 20 with computing unit (μC), driver stages, signal processing such as amplifiers (OP amps), filter circuits, A/D converters and with an associated data memory 21 controls the actuators 4, 41, 42 by means of control lines 84 to carry out ventilation and to carry out an occlusion maneuver 100 (FIG. 2).

To carry out the ventilation and the occlusion maneuver 100 (FIG. 2), measured values from the control unit 20 that are acquired and provided by the sensors 3, 31, 32, 33, 34 are used via signal lines 85. An input element, comprised by an operating and display element (UI, GUI) 23, is connected as a human-machine interface via a data line 83. The operating and display element 23 enables a user to start an occlusion maneuver or a sequence 100 (FIG. 2) or to make settings for carrying out ventilation on the ventilator 1. The operating and display element 23 also serves to provide and/or display measured values or measurement curves of the sensor system 3, events and alarm situations. In addition, an acoustic signal and optional data interfaces can also be arranged in the operating and display element 23.

FIG. 2 shows a schematic sequence 100 of ventilation including occlusion. Identical elements of FIG. 1 and FIG. 2 are labeled with the same reference numbers in FIGS. 1 and 2. The control of this sequence 100 can be carried out, for example, by a control unit 20 as shown in FIG. 1. After a start 101 of ventilation, measured values of an inspiratory pressure level (P_insp.) 104 are continuously acquired in a procedure 102.

Towards the end of an inhalation phase, the occlusion maneuver is started 105—for example initiated by an interaction 103 by a user by means of an operating and display element 23 (FIG. 1)—with an activation 106 of a control of the actuators 4, 41, 42 (FIG. 1) in order bring about a closure (occlusion) of the line system 6, 61, 62 (FIG. 1), of the breathing system 8, 81, 82 (FIG. 1). This leaves the patient 50 (FIG. 1) with only the possibility of gas exchange with the volumes of the duct system 6, 61, 62 (FIG. 1) for the duration of the occlusion.

Subsequently, measured values of the inspiratory pressure level (P_insp.) 107 are continuously acquired in a procedure 108.

At the same time, in the procedure 108, an analysis 110 of the continuously acquired measured values of the inspiratory pressure level (P_insp.) 107 is carried out to determine the inspiratory pressure plateau (P_plat.). As a result of this analysis 110, an indicator 111 is output or provided which indicates the inspiratory pressure plateau (P_plat.).

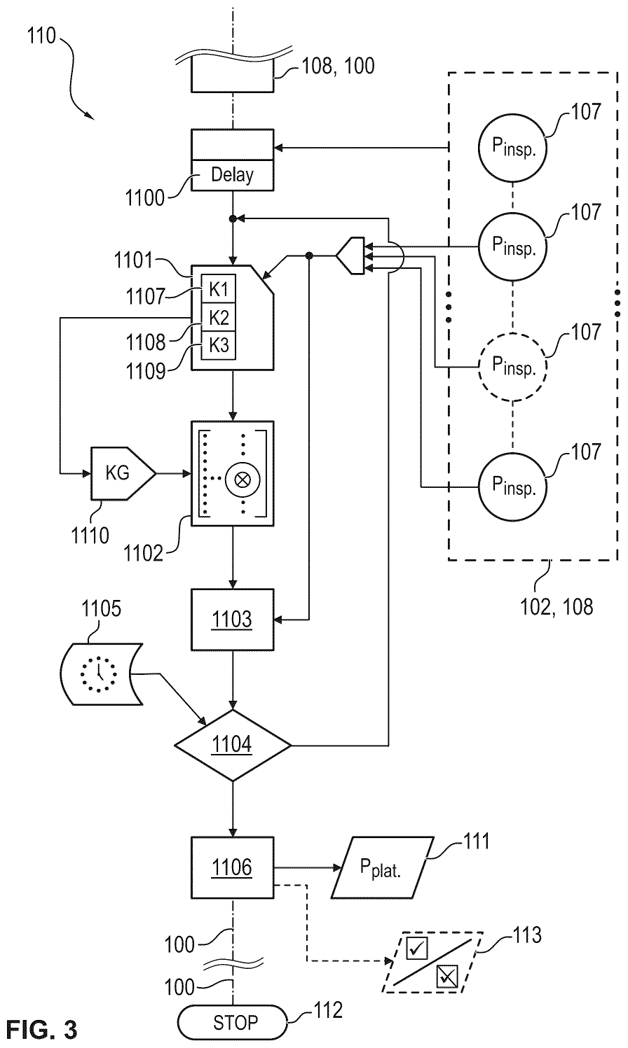

In addition, the analysis 110 determines an optional reliability indicator 113, which can provide an indication of the reliability of the determined inspiratory plateau pressure (P_plat.) 111. With a deactivation 109 of the control of the actuators 4, 41, 42 (FIG. 1), the occlusion reaches an end 112. Subsequently, the ventilation of the patient 50 (FIG. 1) can be continued as before. The performance of the analysis 110 is explained in more detail in some aspects with reference to FIG. 3.

FIG. 3 shows detailed aspects of the schematic sequence 100 according to FIG. 2 with regard to the analysis 110 for determining the inspiratory pressure plateau (P_plat.) 111. Identical elements of FIGS. 1, 2 and FIG. 3 are designated with the same reference numerals in FIGS. 1, 2 and 3. Preferably, an optional waiting time 1100 of, for example, 0.3 seconds to 0.5 seconds is activated after the start of occlusion 108. Subsequently, a signal analysis 1101 is performed as a sub-function of the analysis 110 (FIG. 2) using a statistical methodology as an example, applying a plurality of sectional linear regressions over a signal course (signal curve) of inspiratory pressure measurement values (P_insp.) 107, which are continuously stored as data in an element for data storage 1102.

By applying the sectional linear regressions, which are applied continuously during the duration of the occlusion phase, of approximately 2 seconds for data sets of measured values of the inspiratory pressure (P_insp.) 107, suitable parameters K1, K2, K3 1107, 1108, 1109 are determined, which characterize the course of the measured values of the inspiratory pressure (P_insp.) 107.

Carrying out the linear regression over the duration of the occlusion phase results in a large number of straight line segments, which can be compared, analyzed and interpreted individually or with each other in order to evaluate the course and shape of the inspiratory pressure (P_insp.) 107 and to determine at least one parameter K1, K2, K3 1107, 1108, 1109 on the basis of the data in the data storage element 1102 whether and at what time after activation 106 (FIG. 2) of the occlusion a stable inspiratory pressure plateau (P_plat.) 111 has been established and what pressure level the determined inspiratory pressure plateau (P_plat.) 111 has.

Some possibilities for determining suitable parameters K1, K2, K3 1107, 1108, 1109 when carrying out linear regressions are given here as examples:
a parameter K1 1107, which indicates the gradients of the determined straight line sections;
a parameter K2 1108, which indicates differences between the values approximated by the regression and measured values of the inspiratory pressure (P_insp.) 107;
a parameter K3 1109, which indicates the mean values of the approximated values.
   The parameters K1, K2, K3 1107, 1108, 1109 can—if advantageous also be normalized, inversely normalized and/or weighted for further analysis and simplification. Furthermore, the parameters K1, K2, K3 1107, 1108, 1109 can also be combined in a weighted or unweighted manner in an overall parameter KG 1110. This overall parameter KG 1110 directly and/or indirectly indicates the inspiratory plateau pressure (P_plat.) 111 determined using the parameters K1, K2, K3 1107, 1108, 1109

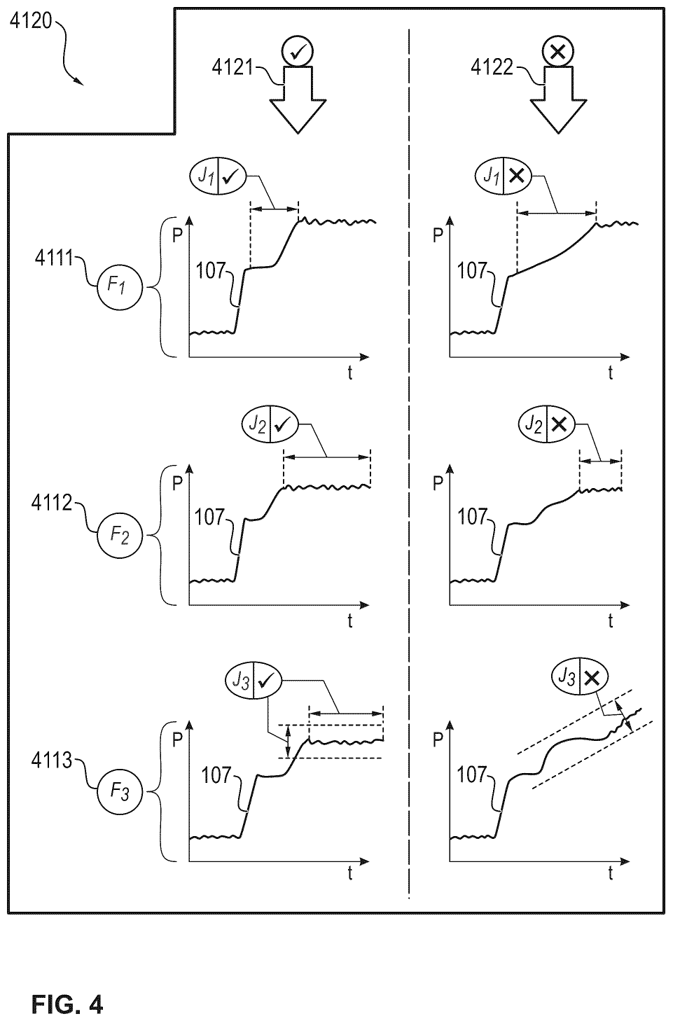

Following the determination of the parameters K1, K2, K3 1107, 1108, 1109, KG 1110, an optional reliability indicator 113 can be determined 1103 on the basis of the measured values of the inspiratory pressure (P_insp.) or the time course of the measured values of the inspiratory pressure (P_insp.)—preferably included in the linear regression process—which can provide an indication of the reliability of the determined inspiratory plateau pressure (P_plat.) 111. For this purpose, the formation of a standard deviation for the inspiratory pressure (P_insp.) 107, starting with the start of the occlusion maneuver 105 (FIG. 2), can be used to evaluate over each regression period in a comparison with threshold values or comparison values whether and/or to what extent the determined inspiratory plateau pressure (P_plat.) 111 is reliable and thus suitable for output to a user. By forming the standard deviation for the inspiratory pressure (P_insp.) 107 over the time course of the occlusion 108 (FIG. 2), a visual evaluation of graphs or curves of the inspiratory pressure (P_insp.) 107—as also shown as an example in FIG. 4 can be simulated in an automated manner and thus objectified and implemented in an advantageous way independent of the user's subjective impressions

Some examples of a visual evaluation of graphs or curves of the inspiratory pressure (P_insp.) are shown in FIG. 4 for illustration.

Following the determination 1103 of the reliability indicator 113, a comparison 1104 is used to check whether a maximum occlusion time duration 1105 has been reached and to what extent the reliability indicator 113 indicates that the determined inspiratory plateau pressure (P_plat.) 111 can be assessed as reliable.

If the duration or the maximum occlusion time duration 1105 has not yet been reached, the acquisition of measured values of the inspiratory pressure (P_insp.) 107 is continued.

If the maximum occlusion time duration 1105 is reached and the reliability indicator 113 indicates that the determined inspiratory plateau pressure (P_plat.) 111 can be assessed as reliable, the occlusion reaches the end 112 (FIG. 2), and a result output 1106 with an output of the inspiratory pressure plateau (P_plat.) 111 is performed. In the result output 1106, an optional output of the optional reliability indicator 113 may also be provided.

Optionally, the sequence check 1106 according to FIG. 3 may provide for premature termination of the occlusion if, for example, an inspiratory pressure plateau (P_plat.) 111 has already been determined with a very high reliability 113 before the maximum occlusion time duration 1105 has elapsed. The transition from this procedure 110 back to the procedure 100 (FIG. 2) then takes place.

FIG. 4 shows aspects of the determination of the inspiratory pressure plateau according to FIGS. 1, 2 and 3. Identical elements of FIGS. 1, 2, 3 and FIG. 4 are labeled with the same reference numbers in FIGS. 1, 2, 3 and 4. FIG. 4 serves to visualize the possibilities of deriving certain characteristics F1 4111, F2 4112, F3 4113 from the signal course of the inspiratory pressure (P_insp.) 107, which can be used to evaluate the quality of the determined inspiratory pressure plateau (P_plat.) 111 (FIG. 3) as an example.

For this purpose, various scenarios 4121, 4122 of possible signal courses of the inspiratory pressure (P_insp.) 107 (FIG. 2, FIG. 3) over time are shown in an unscaled and schematic representation 4120. In the left part of the representation 4120, scenarios 4121 with ideal signal courses and in the right part of the representation 4120, corresponding scenarios 4122 with non-ideal signal courses of the inspiratory pressure (P_insp.) 107 are shown in relation to the three features F1 4111, F2 4112, F3 4113.

Here are some brief explanations of the features F1 4111, F2 4112, F3 4113:
Feature F1 4111 is used to evaluate the maximum rise time (t_rise_max.) of the inspiratory pressure measurement values (P_insp.) 107 after one of the starts of occlusion 105 (FIG. 2);
Feature F2 4112 is used for evaluation with regard to a minimum time duration (t_plat_min.) of a plateau of the inspiratory pressure measurement values (P_insp.) 107 after one of the starts of the occlusion 105 (FIG. 2); and
Feature F3 4113 is used to evaluate the stability (P_stabil.) of the plateau of the inspiratory pressure measurement values ((P_insp.) 107 after one of the starts of occlusion 105 (FIG. 2).

While specific embodiments of the invention have been shown and described in detail to illustrate the application of the principles of the invention, it will be understood that the invention may be embodied otherwise without departing from such principles.

### LIST OF REFERENCE NUMBERS

1 Medical device, ventilator
3 Sensors
4 Actuators
5 Surroundings
6 Line system, breathing tube system
7 Patient connecting element, Y-piece
8 Breathing system
9 Gas supply for oxygen and medical air
20 Control unit
21 Data storage (memory)
23 Input element, operating and display element, UL, GUI
31 Inspiratory flow rate sensor
32 Inspiratory pressure sensor
33 Expiratory flow rate sensor
34 Expiratory pressure sensor
41 Inspiration dosing element
42 Expiratory valve
50 Patient, living being
51, 52 Passive non-return valves
61 Inspiratory ventilation tube
62 Expiratory ventilation tube
81 Inspiratory branch
82 Expiratory branch
83 Data line
84 Control lines, control signals
85 Signal lines, signals, measured values
91, 92 Pneumatic interfaces
100 Procedure, execution of the occlusion maneuver
101 Start carrying out the occlusion maneuver
102, 108 Continuous acquisition of measured values
103 Interaction of the user
104, 107 Measured values of the inspiratory pressure P_insp.
105 Start, beginning of the occlusion maneuver
106 Activation and control of actuators, valves
109 Deactivation and control of actuators, valves
110 Determination of the inspiratory pressure plateau P_plat.
111 inspiratory pressure plateau P_plat.
112 Stop, end of the occlusion maneuver
113 Reliability indicator
1100 Waiting time, Delay
1101 Signal analysis, regression, determination of parameters
1102 Element for data storage (field, array, vector)
1103 Determination of a reliability indicator
1104 Comparison implementation
1105 Maximum occlusion duration
1106 Result output
1107-1110 Parameters K1, K2, K3, KG
4111-4113 Features F1, F2, F3
4120 Presentation of scenarios

What is claimed is:

1. A medical device for ventilating a living being and with an operating function for carrying out an occlusion maneuver to determine an inspiratory pressure plateau, the medical device comprising:
a line system;
a breathing system, which is pneumatically connected to the line system via interfaces, the breathing system comprising a conveying unit for a controlled conveying and supply of an inspiratory amount of a breathing gas mixture to the living being, wherein the line system is configured to guide quantities of the breathing gas mixture to the living being and to guide quantities of the breathing gas mixture away from the living being;
a sensor system, the sensor system comprising at least one pressure sensor which is arranged on the breathing system or on the line system, wherein the sensor system is configured and arranged to continuously detect an inspiratory pressure level or a time course of the inspiratory pressure level and to provide the inspiratory pressure level or the time course of the inspiratory pressure level to the control unit as measured inspiratory pressure values,
a control unit operatively connected to the breathing system and the sensor system and configured to
carry out ventilation of the living being with the conveying unit, the breathing system and the line system by controlling at least one of the predetermined parameters comprising duration of one phase of an inspiration, duration of one phase of an expiration, inspirational pressure, tidal volume, and ventilation frequency,
with the conveying unit, during the phase of an inspiration and/or following the phase of the inspiration to carrying out an occlusion maneuver with a closure of the breathing system for a predetermined time duration of an occlusion phase, wherein for the predetermined time duration of the occlusion phase, no quantities of inspiratory breathing gas mixture pass from the medical device to the living being and no quantities of expiratory breathing gas mixture pass away from the living being,
to determine the inspiratory pressure plateau based on the measured inspiratory pressure values acquired during the occlusion maneuver by means of a signal analysis.

2. A medical device according to claim 1, wherein the control unit is configured to carry out ventilation of the living being with the conveying unit, the breathing system and the line system after the occlusion phase and to generate a display signal during the occlusion phase or during the ventilation, with an indication of the determined inspiratory pressure plateau or with a termination indication that no reliable result of the inspiratory pressure plateau could be expected in the predetermined time duration of the occlusion phase.

3. A medical device according to claim 1, wherein the control unit is configured to include an elasticity of components of the line system in the determination of the inspiratory pressure plateau.

4. A medical device according to claim 1,
wherein the sensor system is configured to continuously detect an expiratory pressure level or a time course of the expiratory pressure level and to provide the expiratory pressure level or the time course of the expiratory pressure level to control unit as expiratory pressure measured values,
wherein the control unit is configured to determine an end-expiratory pressure based on the measured expiratory pressure values, and
wherein the control unit is configured to determine, based on the determined inspiratory pressure plateau and the end-expiratory pressure:
an indicator for a total work of breathing, and/or
an indicator for a share of a spontaneous respiration of the living being of the total work of breathing, and/or an
indicator for a share of the medical device or the ventilator in the total work of breathing, and/or
an indicator for a static distensibility of the lungs of the living being.

5. A medical device according to claim 1, wherein the control unit is configured for determining the inspiratory pressure plateau via the signal course of the inspiratory pressure measured values during the occlusion phase to apply or carry out:
a statistical methodology using a plurality of sectional linear, non-linear or polynomial regressions, or
an analytical process using a continuous determination of a gradient over the signal course of the inspiratory pressure measured values, or
mathematical processes comprising pattern comparison of the inspiratory pressure measured values with comparative data or trend analysis of the inspiratory pressure measured values with comparative data.

6. A medical device according to claim 1,
wherein the control unit is configured to evaluate at least one criterion during the occlusion phase, with the determination of the inspiratory pressure plateau during the occlusion phase,
wherein the at least one criterion comprises:
a maximum time duration of a rise time of the inspiratory pressure measurement values, and/or
a minimum time duration of a plateau of the inspiratory pressure measurement values, and/or
a stability of the plateau of the inspiratory pressure measurement values.

7. A medical device according to claim 1, wherein the control unit is configured to include a reliability indicator in the determination of the inspiratory pressure plateau during the occlusion phase.

8. A medical device according to claim 7, wherein the control unit is configured to terminate the occlusion maneuver based on the verification of the reliability indicator.

9. A medical device according to claim 8, wherein the control unit is configured to generate a display signal to indicate a termination of the occlusion maneuver.

10. A medical device according to claim 1,
wherein the sensor system comprises at least one flow rate sensor, which is arranged on the breathing system or on the line system and is configured to continuously detect at least one measured value of a flow rate and to provide the measured value of flow rate to the control unit,
wherein the at least one flow rate sensor is configured as an inspiratory flow rate sensor, as an expiratory flow rate sensor or as a flow rate sensor positioned adjacent to the patient.

11. A medical device according to claim 1,
wherein an input element is operatively connected to the medical device, which input element is configured to provide a signal to the control unit to cause the control unit to initiate an activation or a start of the occlusion maneuver,
wherein an actuation of the input element by a user causes a closure of the breathing system for a predetermined time interval or maximum time interval or for a duration of the actuation of the input element.

12. A medical device according to claim 1, wherein the control unit is configured to generate a display signal to indicate a time remaining until completion of the occlusion maneuver and/or the occlusion phase.

13. A medical device according to claim 1, wherein the control unit is configured for a defined signal analysis duration with a time window of 0.2 seconds to 0.8 seconds.

14. A process for determining an inspiratory pressure plateau, the process comprising the steps of:
providing a data set which indicates a signal course of inspiratory pressure measured values, wherein the inspiratory pressure measured values correspond to a time duration of an occlusion phase; and
carrying out a signal analysis of the inspiratory pressure measured values to determine the inspiratory pressure plateau.

15. A process according to claim 14, further comprising
providing a data set which indicates a signal course of expiratory pressure measurement values and/or an end-expiratory pressure, and/or acquiring a signal course of measured expiratory pressure values;
subsequently carrying out a signal analysis based on the determined inspiratory pressure plateau and the end-expiratory pressure to determine:
an indicator for a total work of breathing, and/or
an indicator for a share of a spontaneous respiration of the living being in the total work of breathing, and/or
an indicator for a share of the medical device or the ventilator in the total work of breathing, and/or
an indicator for a static distensibility of the lung of the living being.

16. A process according to claim 15, wherein the signal analysis to determine the inspiratory pressure plateau further comprises applying:
a statistical process comprising a plurality of linear sectional regressions, non-linear sectional regressions or polynomial sectional regressions over the signal course of the inspiratory pressure measured values, or
an analytical process with a continuous determination of a gradient over the signal course of the inspiratory pressure measured values over the signal course of the inspiratory pressure measured values, or
a mathematical processes of pattern comparison of the inspiratory pressure measured values with comparative data.

17. A process according to claim 14,
wherein in the determination of the inspiratory pressure plateau a test of at least one criterion is carried out, and the at least one criterion comprises: a maximum time duration of a rise time of the inspiratory pressure measured values, and/or a minimum time duration of the plateau of the inspiratory pressure measurement values, and/or a stability of the plateau of the inspiratory pressure measurement values, and/or
wherein a reliability indicator is included in the determination of the inspiratory pressure plateau, and/or
wherein a determination the inspiratory pressure plateau is further based on properties of the line system.

18. A process according to claim 14, further comprising providing a computer program on a non-transitory computer-readable medium comprising a program code or a non-transitory computer-readable medium computer product comprising the program code, wherein the program code carries out at least some of the steps.

19. A process with an operating function for operating a medical device configured for ventilating a living being with determination of an inspiratory pressure plateau, the process comprising the steps of:
activating a means for controlled conveying of inspiratory and expiratory quantities of breathing gas mixture in order to coordinate ventilation with a cyclical sequence of phases with inspiration and expiration;
activating components of the medical device to effect occlusion of a breathing system for a predetermined time duration of an occlusion phase during an inspiration phase or at the end of an inspiration phase;
providing a signal course or acquiring a signal course of measured pressure values, which indicate an inspiratory pressure level;
carrying out a signal analysis of the inspiratory pressure measured values to determine the inspiratory pressure plateau.

20. A process according to claim 19, further comprising:
providing a data set which indicates a signal course of expiratory pressure measurement values and/or an end-expiratory pressure, and/or acquiring a signal course of measured expiratory pressure values;
subsequently carrying out a signal analysis based on the determined inspiratory pressure plateau and the end-expiratory pressure to determine:
an indicator for a total work of breathing, and/or
an indicator for a share of a spontaneous respiration of the living being in the total work of breathing, and/or
an indicator for a share of the medical device or the ventilator in the total work of breathing, and/or
an indicator for a static distensibility of the lung of the living being.

21. A process according to claim 19, wherein the signal analysis to determine the inspiratory pressure plateau further comprises applying:
a statistical process comprising a plurality of linear sectional regressions, non-linear sectional regressions or polynomial sectional regressions over the signal course of the inspiratory pressure measured values, or
an analytical process with a continuous determination of a gradient over the signal course of the inspiratory pressure measured values over the signal course of the inspiratory pressure measured values, or
a mathematical processes of pattern comparison of the inspiratory pressure measured values with comparative data.

22. A process according to claim 19,
wherein in the determination of the inspiratory pressure plateau a test of at least one criterion is carried out, and the at least one criterion comprises: a maximum time duration of a rise time of the inspiratory pressure measured values, and/or a minimum time duration of the plateau of the inspiratory pressure measurement values, and/or a stability of the plateau of the inspiratory pressure measurement values, and/or
wherein a reliability indicator is included in the determination of the inspiratory pressure plateau, and/or
wherein a determination the inspiratory pressure plateau is further based on properties of the line system.
